(19) United States
(12) Patent Application Publication
(10) Pub. No.: US 2023/0414925 A1
(43) Pub. Date: Dec. 28, 2023

(54) GUIDEWIRE LOADING ASSIST TOOL FOR INTRAVASCULAR BLOOD PUMPS

(71) Applicant: Cardiovascular Systems, Inc., St. Paul, MN (US)

(72) Inventors: Victor L. Schoenle, Greenfield, MN (US); Joseph P. Higgins, Minnetonka, MN (US); Matthew H. Hanson, St. Louis Park, MN (US)

(21) Appl. No.: 17/809,397

(22) Filed: Jun. 28, 2022

(51) Int. Cl. A61M 60/865; A61M 60/178; A61M 60/216; A61M 25/09

(52) U.S. Cl. CPC A61M 60/865; A61M 60/178; A61M 60/216; A61M 25/09041

(57) ABSTRACT

The present invention provides a guidewire assist tool for use in providing a smooth back loading of a guide wire through an intravascular blood pump such as a VAD, LVAD or RVAD. Such devices typically have large apertures or windows, e.g., outlet apertures located proximally of an impeller. The guidewire assist tool temporarily fills the windows to allow the guidewire to smoothly translate past the windows. Once the guidewire tip has translated past the apertures or windows, the guidewire assist tool may be removed.

### CROSS-REFERENCE TO RELATED APPLICATIONS

None

### STATEMENT REGARDING FEDERALLY SPONSORED RESEARCH OR DEVELOPMENT

Not Applicable

### BACKGROUND OF THE INVENTION

### Field of the Invention

The invention relates to a guidewire assist tool for intravascular blood pumps.

### Description of the Related Art

With reference to FIG. 1, the human heart comprises four chambers and four heart valves that assist in the forward (antegrade) flow of blood through the heart. The chambers include the left atrium, left ventricle, right atrium and left ventricle. The four heart valves include the mitral valve, the tricuspid valve, the aortic valve and the pulmonary valve.

The mitral valve is located between the left atrium and left ventricle and helps control the flow of blood from the left atrium to the left ventricle by acting as a one-way valve to prevent backflow into the left atrium. Similarly, the tricuspid valve is located between the right atrium and the right ventricle, while the aortic valve and the pulmonary valve are semilunar valves located in arteries flowing blood away from the heart. The valves are all one-way valves, with leaflets that open to allow forward (antegrade) blood flow. The normally functioning valve leaflets close under the pressure exerted by reverse blood to prevent backflow (retrograde) of the blood.

Thus, as illustrated, the general blood flow comprises deoxygenated blood returning from the body where it is received by the right atrium via the superior and inferior vena cava and is, in turn, pumped into the right ventricle, a process controlled by the tricuspid valve. The right ventricle functions to pump the deoxygenated blood to the lungs via the pulmonary arteries, where the blood is reoxygenated and returned to the left atrium via the pulmonary veins.

Heart disease is a health problem with a high mortality rate. The use of temporary mechanical blood pump devices are used on an increasingly frequent basis to provide short-term acute support during surgery or as temporary bridging support to help a patient survive a crisis. These temporary blood pumps have developed and evolved over the years to supplement the pumping action of the heart on a short-term basis and supplement blood flow as either left or right ventricular assist devices, with the left ventricular assist device (“LVAD”) currently the most commonly used device.

Known temporary LVAD devices generally are delivered percutaneously, e.g., through the femoral artery, to locate or position the LVAD inlet in the patient's left ventricle and the outlet in the patient's ascending aorta with the body of the device disposed across the aortic valve. As the skilled artisan will understand, an incision may be made below the patient's groin to enable access to the patient's femoral artery. The physician may then translate guide wire, followed by a catheter or delivery sheath, through the femoral artery and descending aorta until reaching the ascending aorta. The LVAD with attached rotational drive shaft may then be translated through the delivery catheter or sheath lumen, leaving a proximal end of the drive shaft exposed outside of the patient and coupled with a prime mover such as an electric motor or the equivalent for rotating and controlling the rotational speed of the drive shaft and associated LVAD impeller.

Temporary axial flow blood pumps consist generally of two types: (1) those that are powered by a motor integrated into the device that is connected with the pump's impeller (see U.S. Pat. Nos. 5,147,388 and 5,275,580) ; and (2) those that are powered by an external motor that provides rotational torque to a drive shaft which is, in turn, connected to the pump's impeller (see U.S. Pat. Nos. 4,625,712 to Wampler and U.S. Pat. No. 5,112,349 to Summers, each hereby incorporated by reference in their entirety).

Known temporary ventricle assist devices (“VAD”), including LVAD and RVAD (right ventricular assist) devices, whether with integrated motor or an external motor, generally comprise the following elements mounted within a housing, listed in order from the inflow end to the outflow end: an inflow aperture(s); a stationary inducer, also known as a flow straightener; a rotational impeller; and a stationary diffuser and/or outflow structure; and an outflow aperture(s) as shown in the exemplary prior art pump and/or impeller assembly cross sectional and cutaway view of FIG. 2.

In FIG. 2, the known device 2 is oriented with the inflow end (distal end) on the left side of the drawing and the outflow end (proximal) on the right side, so that the incoming blood flow in the ventricle enters the device housing through the inflow aperture(s) (not shown), flows through the defined by the surrounding housing 14, ultimately entering the impeller/pump assembly 4. There, the incoming blood encounters the stationary inducer 6 before being urged forward by the rotating impeller 8. The blood flow may then be modified by a stationary diffuser and exits into the aorta via the housing's outflow aperture(s) 10.

A guidewire is required to position these blood pump devices within the vasculature. Typically, the guidewire will be back loaded, i.e., initially entering the cannula at the distal tip, then translated proximally through the device. At point, e.g., the large outlet apertures 10 and/or the inlet apertures 12 shown in FIG. 2 may cause difficulties in translating the guidewire.

It would be advantageous to provide a tool that is easily placed, and easily removed, to mitigate the problems presented by the inlet and/or outlet apertures during guidewire loading.

Various embodiments of the present invention address these, inter alia, issues.

The figures and the detailed description which follow more particularly exemplify these and other embodiments of the invention.

### BRIEF DESCRIPTION OF THE SEVERAL VIEWS OF THE DRAWINGS

FIG. 1 is a cutaway view of the human heart;

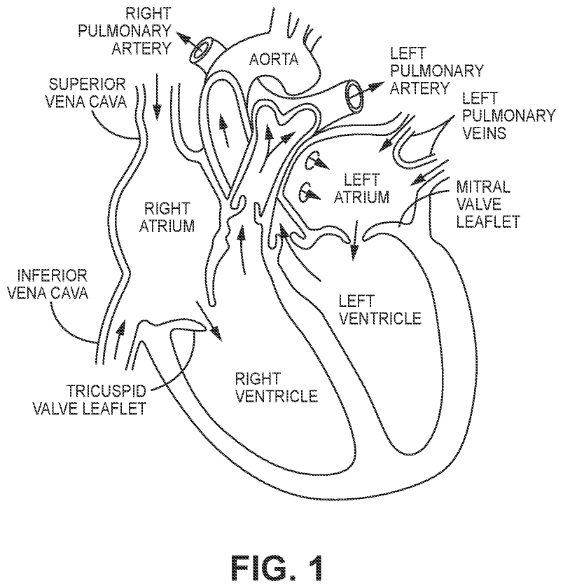

FIG. 2 is a cross-sectional view of a prior art device;

FIG. 3 is a perspective view of one embodiment of the present invention;

FIG. 4 is a perspective view of one embodiment of the present invention;

### DETAILED DESCRIPTION OF THE INVENTION

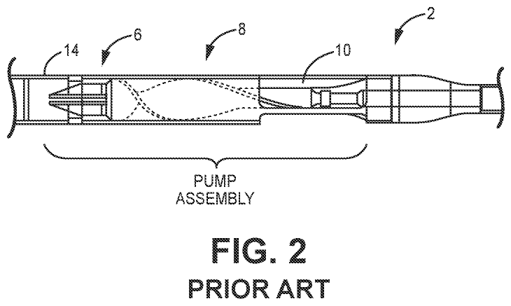

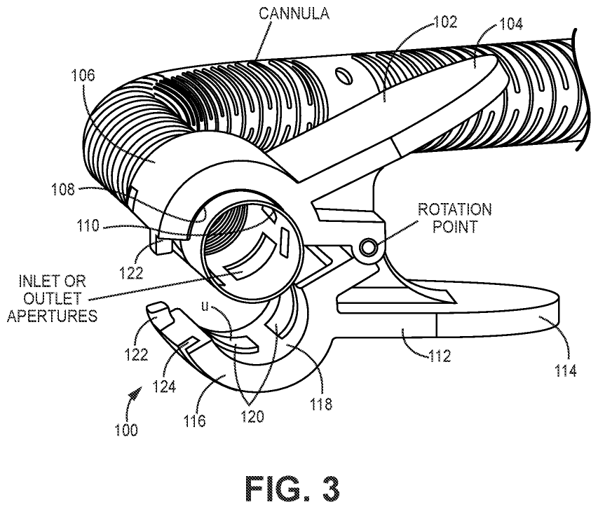

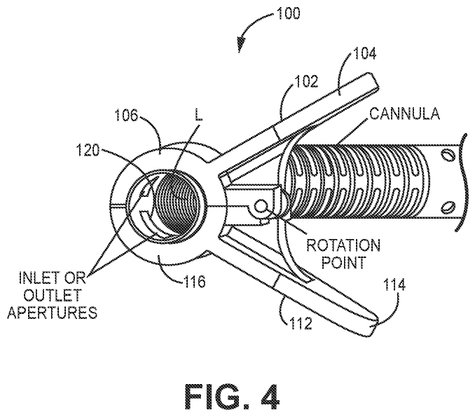

Referring now to FIGS. 3 and 4, and with continued reference to FIG. 2, a cannula defining a lumen and apertures is illustrated. The illustrated apertures may comprise inlet apertures or outlet apertures in a blood pump and the present invention may be used for either inlet or outlet apertures of a blood pump.

FIG. 3 shows an open configuration for the subject guidewire assist tool 100 and FIG. 4 shows the guidewire assist tool 100 in the closed configuration.

The guidewire assist tool 100 comprises a first arm 102 comprising a grip end 104 and a partially circumferential end 106 opposite the grip end 104, the partially circumferential end 106 comprising an inner surface 108 defining at least one raised surface 110, a second arm 112 comprising a grip end 114 and a partially circumferential end 116 opposite the grip end 114, the partially circumferential end 116 comprising an inner surface 118 defining at least one raised surface 120.

As shown in FIGS. 3 and 4, the first and second arms 102, 112 are rotationally connected along a length of the first and second arms 102, 104 at a rotation point and configured such that scissoring the first arm grip end 104 and the second arm grip end 114 toward each other causes the first arm partially circumferential end 106 and the second arm's partially circumferential end 116 to move away from each other and into an open configuration as seen in FIG. 3. In addition, scissoring the first grip end 104 away from the second arm grip end 114 causes the first arm's partially circumferential end 106 and the second arm's partially circumferential end 116 to move toward each other and into the closed configuration of FIG. 4.

The guidewire assist tool 100 may comprise a closure securement mechanism on at least one of the partially circumferential end 106 of the first arm 102 and the partially circumferential end 116 of the second arm 104 to aid in providing a secure connection between the first and second arms 102, 104 at the partially circumferential ends 106, 116 thereof during loading of the guidewire (not shown) into the cannula's lumen L.

Various closure securement mechanisms may be used, and one such mechanism may comprise a magnetic closure disposed between a distal end of the partially circumferential end 106 of the first arm 102 and a distal end of the partially circumferential end 116 of the second arm 104. Thus, when the partially circumferential ends of the first arm and the second arm are rotated or scissored into the closed configuration, the magnetic closure provides a closure force that biases the device in the closed configuration and which requires a threshold force to move to an open configuration. In this manner, the two partially circumferential ends 106, 116 of the first and second arms 102, 104 are held in the closed configuration while the operator may turn to the guidewire loading process.

Another embodiment of a closure securement mechanism may comprise a snap-fit or a friction fit between a distal end of the partially circumferential end 106 of the first arm 102 and a distal end of the partially circumferential end 116 of the second arm 104.

Still another embodiment of a closure securement mechanism may comprise a spring or springs that bias the first and second arms 102, 104 into a closed configuration.

The guidewire assist tool of FIGS. 3 and 4 illustrate a closure securement mechanism 100 that comprises at least one male member 122 extending away from a distal end of one or both of the partially circumferential end 106 of the first arm 102 and/or the partially circumferential end 116 of the second arm 104, and at least one recessed region 124 defined in a distal end of one or both of the partially circumferential end 106 of the first arm 102 and/or the partially circumferential end 116 of the second arm 116, wherein the at least one recessed region 124 is complementary in shape to, and aligned with, the at least one male member 122.

When the at least one male member 122 is rotated into the at least one recessed region 124 during achievement of the closed configuration of the device 100, an engagement between the at least one male member 122 and the at least one recessed region 124 occurs and which requires a threshold force to disengage therefrom. In this manner, the two partially circumferential ends 106, 116 of the first and second arms 102, 104 are held in the closed configuration while the operator may turn to the guidewire loading process and then may be disengaged by applying sufficient force to overcome the threshold force required to disengage the male member(s) 122 from the recessed region(s) 124.

Another embodiment of a closure securement mechanism may comprise a clip or a buckle that operatively clips or buckles the partially circumferential ends 106, 116 of the first arm 102 and the second arm 104 together when the guidewire assist tool 100 is in a closed configuration. Unclipping or unbuckling the closure securement mechanism allows the two partially circumferential ends 106, 116 to rotate away from each other, as described above, to achieve an open configuration.

As discussed above, the guidewire assist tool 100 may further comprise the at least one raised surface 110, 120 defined on the inner surfaces 108, 118 of the partially circumferential ends 106, 116 of the first arm 102 and the second arm 104. The at least one raised surface 110, 120 may comprise a shape that is complementary to a shape, and aligned with the location when the guidewire assist tool 100 is in the closed configuration, of the individual blood pump inlet apertures or outlet apertures, e.g., a rectangle, a square, a polygon or other shape.

The complementary shaping and alignment of the at least one raised surface 110, 120 with the inlet apertures or outlet apertures of a blood pump is configured, when the guidewire assist tool 100 is in the closed configuration of FIG. 4, to engagingly fill and/or block, the blood pump inlet apertures or outlet apertures with the at least one raised surface 110, 120.

In certain embodiments, the at least one raised surface 110, 120 defined by inner surfaces 108, 118 of the partially circumferential ends 106, 116 of the first arm 102 and the second arm 104 may comprise an upper surface U that may be smooth and/or curvilinear.

The curvilinear shaping of the upper surface U of the at least one raised surface 110, 120 may match the curvilinear shaping and radius of the housing of the cannula of the blood pump in the region of the housing at the inlet apertures or the outlet apertures. This matching or mirroring of the curvilinear shaping between the upper surface U of the at least one raised surface 110, 120 and the housing of the cannula allows for a smoothed fit between the at least one raised surface 110, 120 and the inlet or outlet apertures of the cannula when the guidewire assist tool 100 is engaged in a closed configuration as in FIG. 4.

Further, in some embodiments, the at least one raised surface 110, 120 may comprise a height relative to, or rise a distance above, the inner surfaces 108, 118 of the partially circumferential ends 106, 116 of the first arm 102 and the second arm 104. In these embodiments, the cannula housing defining the inlet apertures and/or outlet apertures will comprise a thickness. The height of the at least one raised surface 110, 120 relative to, or the distance that the at least one raised surface 110, 120 rises above, the inner surfaces 108, 118 of the partially circumferential ends 106, 116 of the first and second arms 102, 104 may then be selected to be substantially the same as the housing thickness at the inlet or outlet apertures.

With the above structure, when the guidewire assist tool is in the closed configuration, the at least one raised surface 110, 120 is configured to enable a tip of the guidewire to smoothly translate past the inlet aperture and/or outlet aperture that the at least one raised surface 102, 104 is disposed and/or engaged within.

A significant advantage of the various embodiments of the guidewire assist tool 100, is that an operator can verify operational functionality of the device by operating the blood pump, including impeller, while the guidewire assist tool 100 is in the closed position.

The description of the invention and is as set forth herein is illustrative and is not intended to limit the scope of the invention. Features of various embodiments may be combined with other embodiments within the contemplation of this invention. Variations and modifications of the embodiments disclosed herein are possible and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A guidewire assist tool for loading a guidewire into a blood pump having a housing, inlet apertures defined through the housing, outlet apertures defined through the housing, the guidewire assist tool further comprising:
a first arm comprising a grip end and a partially circumferential end opposite the grip end, the partially circumferential end comprising an inner surface defining at least one raised surface,
a second arm comprising a grip end and a partially circumferential end opposite the grip end, the partially circumferential end comprising an inner surface defining at least one raised surface,
wherein the first and second arms are rotationally connected along a length of the first and second arms and configured such that scissoring the first arm grip end and the second arm grip end toward each other causes the first arm partially circumferential end and the second arm's partially circumferential end to move away from each other and into an open configuration, and
wherein scissoring the first grip end away from the second arm grip end causes the first arm's partially circumferential end and the second arm's partially circumferential end to move toward each other and into a closed configuration.

2. The guidewire assist tool of claim 1, wherein at least one of the partially circumferential end of the first arm and the partially circumferential end of the second arm comprises a closure securement mechanism.

3. The guidewire assist tool of claim 2, wherein the closure securement mechanism comprises a magnetic closure between a distal end of the partially circumferential end of the first arm and a distal end of the partially circumferential end of the second arm.

4. The guidewire assist tool of claim 2, wherein the closure securement mechanism comprises a snap-fit or a friction fit between a distal end of the partially circumferential end of the first arm and a distal end of the partially circumferential end of the second arm.

5. The guidewire assist tool of claim 4, wherein the snap fit or friction fit comprises at least one male member extending away from a distal end of one or both of the partially circumferential end of the first arm and/or the partially circumferential end of the second arm.

6. The guidewire assist tool of claim 4, wherein the snap fit or friction fit comprises at least one recessed region defined in a distal end of one or both of the partially circumferential end of the first arm and/or the partially circumferential end of the second arm, wherein the at least one recessed region is complementary in shape to, and aligned with, the at least one male member.

7. The guide wire assist tool of claim 2, wherein the closure securement mechanism comprises a clip that operatively clips the partially circumferential ends of the first arm and the second arm together when the guidewire assist tool is in a closed configuration.

8. The guidewire assist tool of claim 1, wherein the at least one raised surface defined on the inner surface of the partially circumferential end of the first arm and the second arm are of a shape that is complementary to a shape of the blood pump inlet apertures or outlet apertures.

9. The guidewire assist tool of claim 8, wherein when the guidewire assist tool is in the closed configuration, the at least one raised surface aligns with, and engagingly blocks, the blood pump inlet apertures or outlet apertures.

10. The guidewire assist tool of claim 9, wherein the at least one raised surface defined by inner surfaces of the partially circumferential end of the first arm and the second arm is smooth and curvilinear.

11. The guidewire assist tool of claim 10, wherein the housing of the blood pump at the inlet apertures or the outlet apertures comprises a curvilinear shape and radius, and wherein the curvilinear at least one raised surface matches the curvilinear shape and radius of the blood pump housing at the inlet apertures or the outlet apertures.

12. The guidewire assist tool of claim 10, wherein the at least one raised surface rises a distance above the inner surfaces of the partially circumferential end of the first arm and the second arm.

13. The guidewire assist tool of claim 12, wherein the housing defining the inlet apertures and/or outlet apertures has a thickness and wherein the distance that the at least one raised surface rises above the inner surfaces of the partially circumferential end of the first arm and the second arm is the same as the thickness of the housing at the defined inlet apertures and/or defined outlet apertures.

14. The guidewire assist tool of claim 13, wherein when the guidewire assist tool is in the closed configuration, the at least one raised surface is configured to enable a tip of the guidewire to smoothly translate past the inlet aperture and/or outlet aperture that the at least one raised surface is disposed within.

15. The guidewire assist tool of claim 1, wherein when the guidewire assist tool is in the closed configuration, the at least one raised surface is configured to still allow for free rotation of the impeller with the tool installed.

16. The guidewire assist tool of claim 1, wherein the first arm partially circumferential end and the second arm's partially circumferential end are held closed by a biasing force provided by a spring that is operatively disposed between the first and second arms.
